(19) United States
(12) Patent Application Publication
(10) Pub. No.: US 2024/0315794 A1
(43) Pub. Date: Sep. 26, 2024

(54) A PLATFORM FOR A MAIN CONTROL UNIT

(71) Applicant: 270 SURGICAL LTD., Netanya (IL)

(72) Inventors: Golan SALMAN, Atlit (IL); Hadar SCHWARCZ, Netanya (IL)

(21) Appl. No.: 18/576,549

(22) PCT Filed: Jul. 4, 2022
(86) PCT No.: PCT/IL2022/050710

§ 371 Date: Jan. 4, 2024

Related U.S. Application Data
(60) Provisional application No. 63/218,575, filed on Jul. 6, 2021.

(51) Int. Cl. A61B 50/24; A61B 50/20

(52) U.S. Cl. CPC A61B 50/24; A61B 2050/21

(57) ABSTRACT

Provided herein are platforms having magnetic regions for securely holding main control unit of a medical instrument and systems including the same. Further provided are systems including a platform having magnetic regions for holding and securing a main control unit of an endoscope during endoscopic procedures.

### TECHNICAL FIELD

The present disclosure relates generally to a platform having magnetic regions for securely holding a main control unit of a medical device and systems utilizing the same. In particular, the disclosure relates to a platform unit for safely holding and securing a main control unit of an endoscope.

### BACKGROUND

The operation of various medical devices is controlled by a main control unit (MCU). The main control unit generally includes various parts and elements, including, connection interfaces, control panel, processors, displays and the like. In many cases, the MCU connects to the medical device via a detachable dedicated connector which allow the physical and functional association between the medical device and the main control unit.

An endoscope is a medical device used to image an anatomical site (e.g., an anatomical/body cavity, a hollow organ). Unlike some other medical imaging devices, the endoscope is inserted into the anatomical site (e.g., through small incisions made on the skin of the patient). An endoscope can be employed not only to inspect an anatomical site and organs therein (and diagnose a medical condition in the anatomical site) but also as a visual aid in surgical procedures. Medical procedures involving endoscopy include, for example, laparoscopy, arthroscopy, cystoscopy, ureterostomy, hysterectomy and others.

During operation, the endoscope is held and maneuvered by a user while being physically and/or functionally connected to the main control unit. The connection between the endoscope and the main control unit should be uncompromised so as not to affect smooth operation of the endoscope and should also not physically interfere with the manual maneuvering thereof by the user.

There is thus a need in the art for platform/holder of main control unit of a medical device that can securely and safely hold the main control unit in a correct position, and which further allows access to easily and securely connect the medical device (physically or wirelessly) or to the main control unit, while allowing safe and uninterrupted operation of the medical device, during the medical procedure.

### SUMMARY

Aspects of the disclosure, according to some embodiments thereof, relate to an advantageous platform/holder of a main control unit of a medical device, wherein the platform includes at least one magnetic region configured to allow a secure placement and holding of the main control unit during, before or after a medical procedure as operating of the medical device to allow uncompromised (uninterrupted) operation of the medical device. According to some embodiments, the platform is capable of holding/securing the main control unit at a correct and convenient spatial location to allow easy access to panels of the MCU and allow connecting/disconnecting the medical device from the MCU, and connecting/disconnecting the hospital/clinic systems from the MCU, in a secure and controllable manner. In some embodiments, the platform is mounted on/associated with a holding arm, capable of holding the platform at a desired orientation/angle. In further embodiments, the platform is mounted on a cart, thereby advantageously allowing securing the MCU to the cart.

According to some embodiments, the advantageous platform includes one or more grooves, at least one of the grooves includes a magnetic region that are configured to interact/associate with corresponding supporting elements (such as, legs) of the MCU, to thereby facilitate the supporting and holding of the MCU before and/or during the medical procedure. In some embodiments, the advantageous platform allows connecting/disconnecting cables and other elements from the MCU, before, during and/or after operation, without inducing movement of the MCU, thereby improving operation and maneuverability of the medical device by a user. This is of particular importance with hand-held medical devices, such as, endoscopes, which are connected at their proximal end to the main control unit (for example, the handle of the endoscope may be connected to the MCU via a connector of a utility cable having a corresponding socket on a panel of the MCU) while a distal tip thereof is maneuvered within a body or tissue region. Utilizing an advantageous platform as disclosed herein allows securing of the MCU to the platform (thereby ensuring that the MCU does not move, for example, when the endoscope is connected thereto and/or during operating (maneuvering of the endoscope), while maintaining flexibility and freedom of operation to the user under various operating settings and scenarios.

According to some embodiments, there is provided a platform for supporting a main control unit (MCU) of a medical device, the platform includes a surface having one or more grooves, at least one of the grooves includes a magnetic region, wherein the grooves are configured to associate with supporting elements of the MCU.

According to some embodiments, the surface of the platform is essentially flat.

According to some embodiments, the number of grooves corresponds to the number of the supporting elements of the MCU.

According to some embodiments, the association between the grooves and the supporting elements facilitate securing and holding of the MCU to the platform, such that connecting and disconnecting elements from the MCU essentially do not induce movement of the MCU.

According to some embodiments, the position, shape and/or size of the grooves are predetermined.

According to some embodiments, the position, shape and/or size of the grooves are adjustable.

According to some embodiments, the platform may further include a supporting member configured to attach or engage the platform to a holding arm.

According to some embodiments, the supporting member is attached or mounted on a cart by a mounted arm.

According to some embodiments, the height of the supporting member and/or the mounted holding arm is adjustable.

According to some embodiments, at least one of the supporting elements of the MCU is or includes a magnet or magnetizable material.

According to some embodiments, at least one of the supporting elements includes a soft material.

According to some embodiments, the size and/or shape of the supporting elements may be identical.

According to some embodiments, the size and/or shape of the supporting elements may be different.

According to some embodiments, the platform is configured to facilitate placement and securing of the MCU to the platform in a reversible manner.

According to some embodiments, the platform is configured to allow floating of the MCU front panel, to thereby allow access to the front panel.

According to some embodiments, the platform is configured to facilitate uninterrupted physical connection of the medical device to the MCU.

According to some embodiments, the platform is configured to facilitate un-interpreted operation of the medical device during a medical procedure, while the medical device is connected to the MCU.

According to some embodiments, the medical device is an imaging device.

According to some embodiments, the imaging device includes an endoscope.

According to some embodiments, an operating handle of the endoscope is configured to physically connect to the front panel of the MCU while the MCU is placed on the platform.

According to some embodiments, the operating handle is connected to the front panel of the MCU using a connector of a utility cable.

According to some embodiments there is provided a system for supporting a main control unit (MCU) of an endoscope, the system includes the platform as disclosed herein; and the main control unit of an endoscope.

According to some embodiments of the system, the platform is mounted on/attached to a cart.

According to some embodiments, the MCU includes a socket configured to connect to a proximal handle of the endoscope via a connector.

According to some embodiments, the MCU includes four supporting elements.

According to some embodiments, the platform includes four grooves corresponding in position and size to the supporting elements of the MCU.

According to some embodiments, at least one of the supporting elements of the MCU is or includes a magnet or magnetizable material, configured to at least partially interact with a corresponding magnetic region of a corresponding groove on the surface of the platform.

According to some embodiments, at least one of the supporting elements includes a soft material.

According to some embodiments, the size and/or shape of the supporting elements of the MCU is identical. According to some embodiments, the size and/or shape of the supporting elements is different.

According to some embodiments of the system, the surface area of the platform is smaller than the surface area of the MCU, thereby allowing free access to the front panel of the MCU.

According to some embodiments, the system is configured to facilitate placement and securing of the MCU to the platform in a reversible manner.

According to some embodiments, the system is configured to allow floating of the MCU front panel, to thereby allow access to the front panel.

According to some embodiments, the system is configured to facilitate uninterrupted physical connection of the medical device to the MCU.

According to some embodiments, the system is configured to facilitate interpreted operation of the medical device during a medical procedure, while the medical device is connected to the MCU.

According to some embodiments, the endoscope may include at least two cameras at a distal tip thereof. According to some embodiments, the at least two cameras comprise a front camera and a first side-camera. According to some embodiments, the at least two cameras further include a second side-camera, wherein the first side-camera and the second side-camera are positioned on opposite sides of the endoscope tip, and wherein the first side-camera is positioned distally relative to the second side-camera.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIGS. 1A-B schematically depict perspective views of a main control unit situated on a magnetic platform, according to some embodiments;

FIG. 2 illustrates a side view of a main control unit situated on a magnetic platform, according to some embodiments;

FIG. 3 illustrates a front view of a main control unit situated on a magnetic platform, according to some embodiments;

FIG. 4 illustrates a top view of a main control unit situated on a magnetic platform, according to some embodiments;

FIG. 5 illustrates a perspective view of a magnetic platform, according to some embodiments;

FIG. 6 illustrates a side view cross-section of a magnetic platform, according to some embodiments;

FIG. 7 illustrates a top view of a magnetic platform, according to some embodiments;

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

The present invention is directed to an advantageous platform having one or more magnetic regions, configured to hold, stabilize and effectively secure a main control unit of a medical device, thereto. The main control unit is configured to physically connect to the medical device, for example, by a suitable connector of a utility cable. The main control unit may be of a medical imaging device, such as, for example, an endoscope.

As used herein, the terms, “platform”, “holder”, “shelf”, “tray”, “frame” may interchangeably be used. The terms relate to a surface having at least one magnetic region, for holding and securing a main control unit (MCU) of a medical device. In some embodiments, the platform allows securely holding the MCU while allowing secure and easy access to panels of the MCU, without compromising the operation of the medical device. In some exemplary embodiments, the MCU may be an MCU as disclosed in patent application international publication No. WO 2021/024245 to the present applicant.

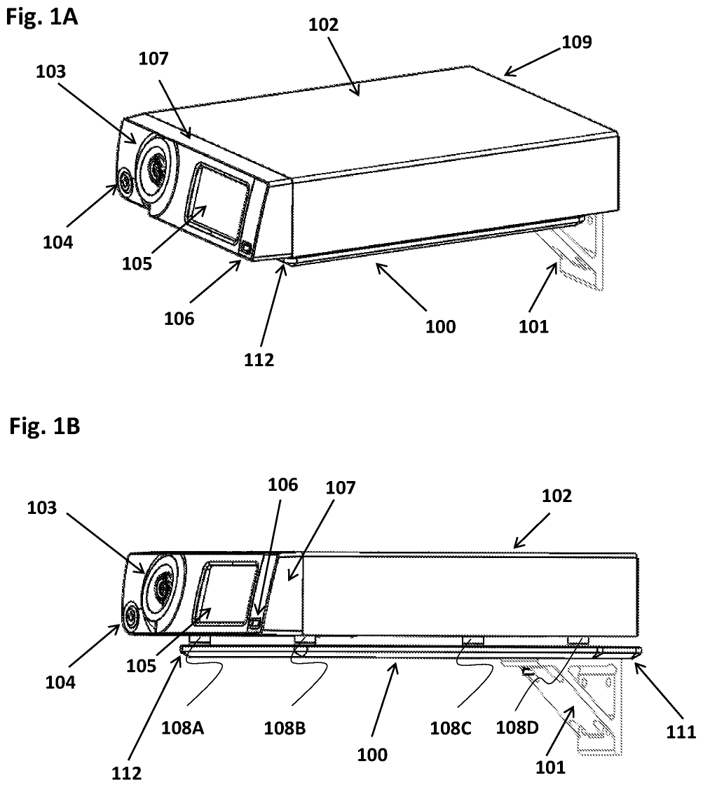

Reference is now made to FIGS. 1A-B, which schematically depict perspective views of a main control unit 102 situated on a platform 100, according to some embodiments. As shown in FIG. 1A, main control unit 102, which is a main control unit of a medical device (such as a medical imaging system, not shown), may include a front panel 107. Front panel 107 may include a connector port 103 (e.g., for connecting to a medical device, such as, for example, an endoscope), a push member 104 (e.g., on/off button), user control interface 105 (e.g., buttons and/or knobs, a touch panel, a touch screen) and may also include a connecting port (e.g., universal serial bus (USB) connector) 106 that may be further connected to an electronic device or implemented as data transferring port. Main control unit 102 may include back panel 109 that may include back ports (for example, USB ports, display ports, high-definition media interface (HDMI) ports, alternating current (AC) power port, and the like (not shown)).

Platform 100 is configured to mount on a supporting member 101, that may further attach platform 100 to a portable pole, holding arm, which may be stationary and/or attached/mounted to, for example, a cart (not shown). Supporting member 101 may be removably or permanently attached to platform 100 and/or to the portable pole/arm/cart. According to some embodiments, supporting member 101 and platform 100 may be integrally manufactured as one unit. According to some embodiments, supporting element 101 may be configured to allow folding of platform 100 (for instance, during storage time), and/or exhibit adjustable properties (for instance, height or angle relative to the portable pole/arm/cart to facilitate the accessibility to the main control unit).

FIG. 1B depicts side-front view, which illustrates a control unit 102 that may include one or more (a plurality of) supporting elements, shown in FIG. 1B as exemplary supporting elements 108A, 108B, 108C, 108D. Exemplary supporting elements 108A-D exhibit at least at portions thereof magnetic properties, (i.e., magnets or magnetized materials (materials that are attracted by a magnetic force). In some embodiments, the supporting elements 108 may be completely made of magnets or magnetized materials. In some embodiments, at least a portion of the supporting elements 108 may be made of magnet or magnetised materials. In some embodiments, at least a portion of the magnetic elements may be coated by magnetized materials. In some embodiments, the supporting elements 108 may be detachably or permanently mounted/placed/secured to a bottom part of a main frame of main control unit 102 for an improved retaining, positioning and/or securing to a platform 100, as detailed herein. In some embodiments, the supporting elements 108 may be identical, similar or different with respect of size, shape and/or composition. In some embodiments, the geometry of the supporting elements 108 may be cylindrical, rectangular, triangular, and the like. In some embodiments, the supporting elements 108 may further include a soft coating on at least a portion thereof. In some embodiments, the supporting elements 108 may at least partially include rubber coating.

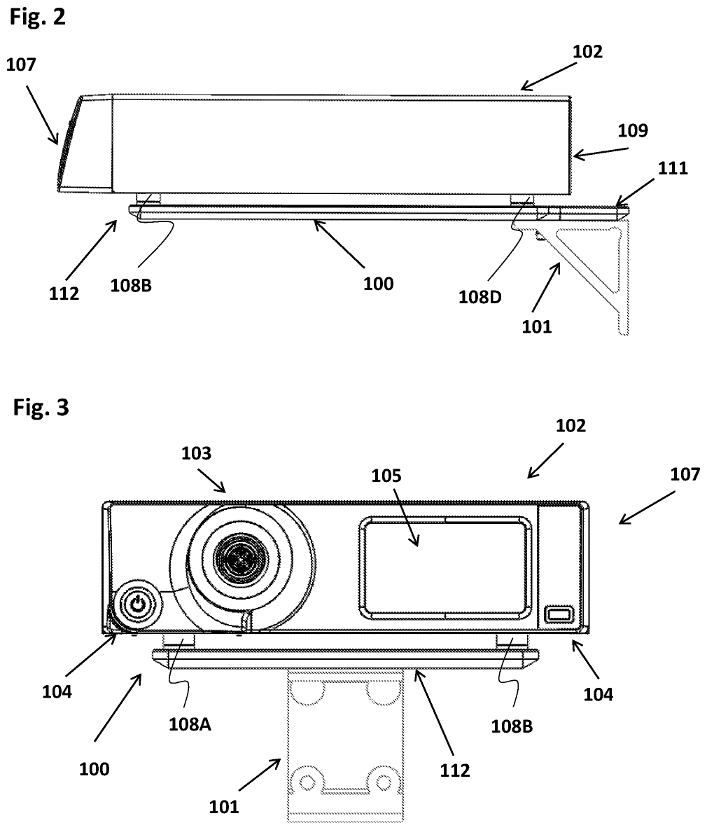

Reference is now made to FIG. 2, which schematically illustrates a side view of a main control unit 102 situated on a platform 100, according to some embodiments. As shown in FIG. 2, main control unit 102 is mounted on platform 100, via supporting elements 108A-D (supporting elements 108A and 108C are not shown), which are magnetically engaged with platform 100. According to some embodiments, main control unit 102 may be retained/placed/positioned on platform 100 wherein a front panel 107 of main control unit 102 is floating and is not fixed/engaged with platform 100, thereby allowing easy access to a connector port 103 (not shown), push member 104 (not shown), user control interface 105 (not shown) and/or other regions/elements of front panel 107.

Reference is now made to FIG. 3 which schematically illustrates a front view of a main control unit 102 situated on a platform 100, according to some embodiments. As shows in FIG. 3, the dimensions of platform 100 may differ from the dimensions of main control unit 102 and/or from a front panel 107 of main control unit 102. For instance, front panel 107 and/or main control unit 102 may be wider than platform 100, without decreasing the stability or the security of a magnetic attraction between platform 100 and main control unit 102, allowing high accessibility to front panel 107 during a medical procedure. Front panel 107 may include a connector port 103 (e.g., for connecting to a medical device, such as, for example, an endoscope), a push member 104 (e.g., on/off button), user control interface 105 (e.g., buttons and/or knobs, a touch panel, a touch screen) and may also include a connecting port (e.g., universal serial bus (USB) connector) 106 that may be further connected to an electronic device or implemented as data transferring port. According to some embodiments, surface area of main control unit 102 is larger than surface area of platform 100. FIG. 3 shows an exemplary illustration wherein a supporting element 101 is attached to the lower surface 112 of platform 100. According to other embodiments, supporting element 101 may be attached to platform 100 through other surfaces, such as top or side surfaces. According to other embodiments, supporting element 101 may be further mounted on a portable pole/arm/cart (not shown) through other surfaces, such as top or side surfaces.

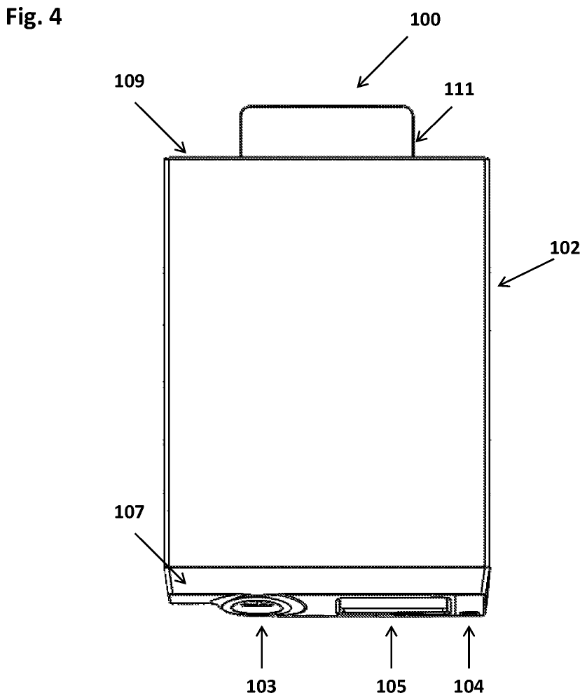

Reference is now made to FIG. 4 which schematically illustrates a top view of a main control unit 102 situated on a platform 100. According to some embodiments, front panel 107 of main control unit 102 may appear floating (positioned outwardly with respect to platform 100), while a back panel 109 of main control unit 102 may be partially or fully supported by a back end 111 of platform 100. According to some embodiments, back end 111 of platform 100 may be extended (positioned outwardly with respect to back panel 109 of main control unit 102) for attaching supporting element 101 to platform 100 and/or enabling proper connections back ports (not shown, for example for connecting cables of devices) to back panel 109.

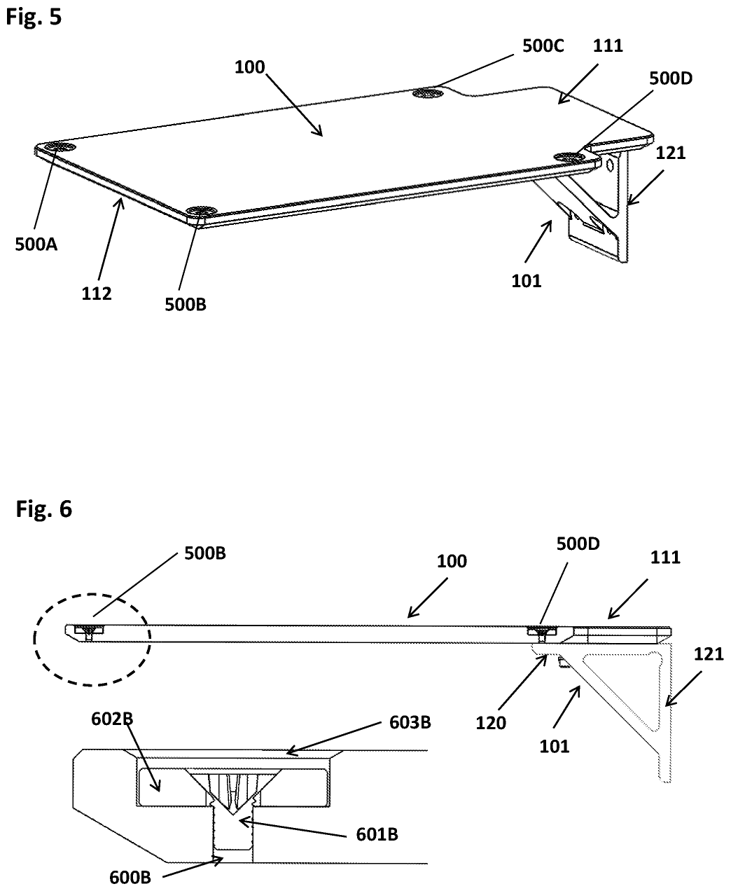

Reference is now made to FIG. 5 which shows a perspective view of a platform 100, according to some embodiments. Shown in FIG. 5 are grooves 500A, 500B, 500C and 500D which are configured to interact (i.e., engage, hold, secure) with corresponding supporting elements 108A-D of a main control unit 102 (as shown in FIGS. 1B-3). According to some embodiments, presence of such grooves may improve the securing of main control unit 102 onto platform 100, in particular before, during and/or after the operation of a medical device (not shown), including, for example: (1) before and/or after connecting the medical device (such as an endoscope) to its respective a connector port 103; (2) while connecting/disconnecting the medical device (such as, an exemplary endoscope) to main control unit 102 (an action which might require an active force from a user); (3) during a medical procedure which requires mobility of main control unit 102 and platform 100 thereof. In some embodiments, the grooves 500 may be identical, similar or different with respect of size, shape and/or composition. In some embodiments, the geometry of the grooves 500 may be cylindrical, rectangular, triangular, and the like.

According to some embodiments, a supporting element 101 may be adjustable and attached to platform 100 by different angles, such as 90° (as depicted in FIG. 5), 110°, and the like. Supporting member 101 may be removably or permanently attached to platform 100 by holding arm 120 (not shown) and/or to a portable pole/arm/cart (not shown) by a mounted arm 121. According to some embodiments, an angle between holding arm 120 and mounted arm 121 may be adjustable by different angles, such as 90° (as depicted in FIG. 5), 110°, 80° and the like. According to some embodiments, supporting member 101 and platform 100 may be integrally made as one unit. According to some embodiments, supporting element 101 may be configured to allow folding of platform 100 (for instance, during storage time), and/or exhibit adjustable properties (for instance, height fixing on the portable pole/arm/cart to facilitate the accessibility to the main control unit 102).

Reference is now made to FIG. 6 which schematically illustrates a side view cross-section of a platform 100, according to some embodiments. Shown in FIG. 6 are exemplary grooves 500B and 500D. For a detailed illustration, a cross-section enlarged view of groove 500B is also presented. As shown in FIG. 6, exemplary groove 500B may include a slot/aperture 600B (e.g., a drilled hole, a housing), a securing element 601B (e.g., a screw, a bolt, removably or permanently attached) and a magnetic element 602B. In some embodiments, securing element 601B is configured to be fixed inside aperture 600B with magnetic element 602B. To maintain tight contact and reduce loosening of magnetic element 602B and/or securing element 601B, an adhesive or a soft material (not shown) may be applied into aperture 600B. According to some embodiments, inward position of fixed magnetic element 602B may result in formation of a pore/opening/groove 603B on the surface of platform 100. The dimensions of pore/opening 603B may be adjusted to fit or correspond to the dimensions of the respective supporting element 108B of a main control unit 102 (as shown in FIGS. 1B-3), for providing additional mechanical stabilization to main control unit 102, while being placed/positioned/secured on platform 100. According to other embodiments, securing element 601B and magnetic element 602B may be made as one unit. According to other embodiments, platform 100 and grooves 500A-D may be integrally manufactured as one unit.

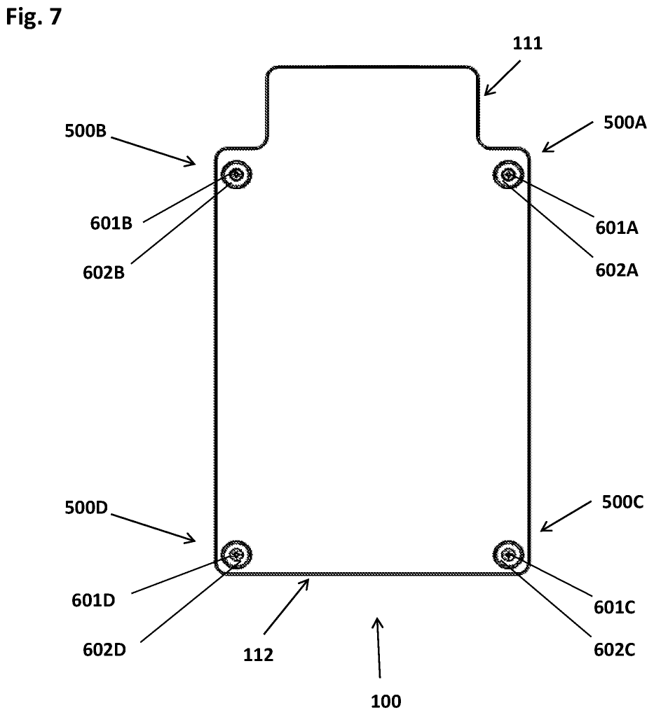

Reference is now made to FIG. 7, which illustrates a top view of a platform 100 having grooves 500A-D, securing elements 601A-D and magnetic elements 602A-D. According to some embodiments, the positions of supporting elements 108A-D of a main control unit 102 (as shown in FIGS. 1B-3) are adapted to the positions of grooves 500A-D of platform 100. In some embodiments the location/position of the grooves 500 along the platform 100 is predetermined. In some embodiments, the location/position of the grooves 500 along the platform 100 are adjusted, according to the MCU used. In some embodiments, the size, relative location, depth, composition, and the like between the grooves 500 on the platform 100 may be identical, similar or different. In some embodiments, the grooves 500 maybe symmetrically located on the face/surface of the platform 100. In some embodiments, the grooves 500 are distributed in a pattern maximizing the distance between each of the grooves 500 for increasing the stability and the magnetic attraction of the MCU 102 to the platform 100. FIG. 7 shows exemplary platform 100 wherein the dimensions of a front end 112 of platform 100 differ from the dimension of a back end 111 of platform 100. In some embodiments, the geometry of the front end 112 of platform 100 may differ from the back end 111 thereof.

According to some embodiments, the platform is disposable. In some embodiments, the platform is reusable. In some embodiments, the platform is sterilizable. In some embodiments, the platform is autoclavable. In some embodiments, the platform is custom made to a specific MCU. In some embodiments, the platform is adjustable to fit various types and/or sizes of MCU.

According to some embodiments, surface area of the platform may be smaller, larger or identical to the surface area of the MCU. Herein, the term “surface area” is referred to the bottom (lower) surface area of the MCU, and to the top (upper) surface area of the platform. The dimensions of the surface area and the volume of the entire platform, as well as its geometry, may depend on the weight, size and/or shape of the MCU to be supported thereby. In some exemplary embodiments, the surface area of the platform is at least 85% smaller than the surface area of the MCU. In some exemplary embodiments, the surface area of the platform is at least 75% smaller than the surface area of the MCU. In some exemplary embodiments, the surface area of the platform is at least 65% smaller than the surface area of the MCU.

In some embodiments, the length of the platform may be smaller, larger or identical to the length of the MCU. In some exemplary embodiments, the length of the platform is at least 85% smaller than the length of the MCU. In some exemplary embodiments, the length of the platform is at least 75% smaller than the length of the MCU. In some exemplary embodiments, the length of the platform is at least 65% smaller than the length of the MCU.

In some embodiments, the width of the platform may be smaller, larger or identical to the width of the MCU. In some exemplary embodiments, the width of the platform is at least 85% smaller than the width of the MCU. In some exemplary embodiments, the width of the platform is at least 75% smaller than the width of the MCU. In some exemplary embodiments, the width of the platform is at least 65% smaller than the width of the MCU.

In some embodiments, the geometry of the platform may be circular, oval, triangular, rectangular, polygonal, and the like, or a combination of several geometries. In some embodiments, the platform does not attach, does not interact, or does not physically touch with the front panel of the MCU, enabling full user access to the front panel, which is essential during performing a medical procedure, particularly during an invasive medical procedure, such as endoscopy.

In some embodiments, the back panel of the MCU may be positioned inwards with respect to platform edge, revealing an uncovered/excessive portion of the platform. In some embodiments, the uncovered portion of the platform may be beneficially implemented for installing the platform onto a portable pole/arm/cart. In some embodiments, a gap may be present between the back end of the platform and the portable arm/pole that may be required for connecting devices and/or connectors and/or cables (for example AC cable, a monitor, and the like) to the back panel of the MCU.

In some embodiments, the surface of the platform may be flat and rigid. In some embodiments, the platform may have rough, smooth, or any combination of surfaces. In some embodiments, rough surface might be advantageous to improve the stability of the MCU on the platform, by providing additional friction to the associated portions thereof.

In some embodiments, the platform may be made of various types of materials, including metals, composite materials, stainless steel, plastics, wood, and the like, depending on the weight, size and/or shape of the MCU to be supported thereby.

In some embodiments, the platform may possess one or more surface coatings exhibiting different material properties than the bulk of the platform, for example, a magnetic surface coating added to a non-magnetic bulk material of the platform. The surface coating may be homogeneous, i.e., possessing uniform coating properties, or heterogeneous, i.e., including several regions with different properties, such as a non-magnetic coating including magnetic regions. The coating of the platform may be fully continuous, partly continuous, i.e., covering only a portion of the surface, or discontinuous.

According to some embodiments, grooves present on the platform may exhibit different, similar or identical composition as the platform, for example, magnets or magnetized materials (materials that are attracted by a magnetic force), or non-magnetic materials. In some embodiments, grooves present on the platform may exhibit different, similar or identical composition as the coating of the platform, for example, magnetic coating.

In some embodiments, grooves present on the platform may include locking mechanism coupled to the MCU supporting elements, wherein the locking mechanism is configured to retain the MCU on the platform. In some embodiments, the MCU unit may slide on the platform until reaching the grooves, wherein the grooves or the locking mechanism of the grooves secure the platform at a predetermined position on the platform.

In some embodiments, the grooves may include one or more brackets, ridges, budges, ledges, or the like, configured to interact with the corresponding supporting elements of the MCU, whereby upon such interaction, the MEU may be secured onto the platform. In some embodiments, the grooves may include at least portions thereof non-smooth surfaces, configured to interact with the corresponding supporting elements of the MCU and prevent movement/sliding thereof on the platform. In some embodiments, the grooves may include at at least a region thereof a Velcro lining, configured to interact with the supporting elements of the MCU, thereby securing the MCU onto the platform.

In some embodiments, the distribution of the grooves position on the platform is designed to increase the balancing of the MCU on the platform, for example, by increasing or decreasing the distance between each of the grooves.

In some embodiments, presence of one groove may be sufficient for stabilizing and securing the MCU on the platform. If only one groove is present on the platform, it may be beneficial to design the groove in various non-circular geometries (not circular or cylindrical or the like) to avoid a possible rotation of the MCU on the platform. In some embodiments, wherein the platform includes a plurality of grooves, various types of grooves geometries may be present on the platform.

In some embodiments, fully or partially rectangular geometry of the platform may possess 4 grooves, positioned essentially at the corners of the platform or in close proximity thereto. In some embodiments, the grooves may be symmetrically distributed on the platform.

In some embodiments, implementing different types of grooves (for example, with respect of composition, shape, size, and the like) may facilitate support of the MCU on the platform. For example, various grooves may include different magnetizing materials. For example, only a portion of the grooves to exhibit magnetic properties.

In some embodiments, depth (height) of the grooves may vary and is configured to correspond to the length (height) of the MCU supporting elements. For instance, the height of the grooves may be about 5% of the height of the MCU supporting elements, height of the grooves may be about 10% of the height of the MCU supporting elements, height of the grooves may be about 20% of the height of the MCU supporting elements, height of the grooves may be about 50% of the height of the MCU supporting elements, height of the grooves may be about 100% of the height of the MCU supporting elements. Any additional height ratios between the grooves depth (height) and the height of the MCU supporting elements are possible, in the range of 5% up to about 100%.

In some embodiments, the platform may be positioned perpendicularly to a portable pole/arm/cart (angle of 90°, i.e., parallel to the floor) or to be adjustable, enabling variety of positioning angles (for example, in the range of 80-150 degrees), for increasing user's accessibility and/or improving holding/securing of the MCU to the platform.

In some embodiments, the platform may be installed on the portable arm/pole at adjustable heights, according to the preference of the user, the MCU used and/or the requirements of the performed medical procedure.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 99% and 101% of the given value.

As used herein, according to some embodiments, the terms “substantially” and “about” may be interchangeable.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. A platform for supporting a main control unit (MCU) of a medical device, the platform comprises a surface comprising one or more grooves, at least one of the grooves comprises a magnetic region, wherein the grooves are configured to associate with supporting elements of the MCU.

2. The platform of claim 1, wherein the surface of the platform is essentially flat.

3. The platform of claim 1, wherein the number of grooves corresponds to the number of the supporting elements of the MCU.

4. The platform according to claim 1, wherein the association between the grooves and the supporting elements facilitate securing and holding of the MCU to the platform, such that connecting and disconnecting elements from the MCU essentially do not induce movement of the MCU.

5. The platform according to claim 1, wherein the position, shape and/or size of the grooves are predetermined.

6. The platform according to claim 1, wherein the position, shape and/or size of the grooves are adjustable.

7. The platform according to claim 1, further comprising a supporting member configured to attach or engage the platform to a holding arm, wherein the supporting member is attached or mounted on a cart by a mounted arm and wherein the height of the supporting member and/or the holding arm is adjustable.

8. (canceled)

9. (canceled)

10. The platform according to claim 1, wherein at least one of the supporting elements of the MCU is or comprises a magnet or magnetizable material.

11. The platform according to claim 1, wherein at least one of the supporting elements comprises a soft material.

12. (canceled)

13. (canceled)

14. The platform according to claim 1, configured to facilitate placement and securing of the MCU to the platform in a reversible manner.

15. The platform according to claim 1, configured to allow floating of the MCU front panel, to thereby allow access to the front panel.

16. (canceled)

17. (canceled)

18. The platform according to claim 1, wherein the medical device is an endoscope and wherein an operating handle of the endoscope is configured to physically connect to the front panel of the MCU while the MCU is placed on the platform.

19. (canceled)

20. (canceled)

21. (canceled)

22. A system for supporting a main control unit (MCU) of an endoscope, the system comprising:
a platform for supporting a main control unit (MCU) of an endoscope, the platform comprises a surface comprising one or more grooves, at least one of the grooves comprises a magnetic region, wherein the grooves are configured to associate with supporting elements of the MCU; and
the main control unit of an endoscope.

23. The system of claim 22, wherein the platform is mounted on/attached to a cart.

24. The system of claim 22, wherein the MCU comprises a socket configured to connect to a proximal handle of the endoscope via a connector.

25. The system according to claim 22, wherein the MCU comprises four supporting elements and wherein the platform comprises four grooves corresponding in position and size to the supporting elements of the MCU.

26. (canceled)

27. The system according to claim 25, wherein at least one of the supporting elements of the MCU is or comprises a magnet or magnetizable material, configured to at least partially interact with a corresponding magnetic region of a corresponding groove on the surface of the platform.

28. (canceled)

29. (canceled)

30. (canceled)

31. The system of claim 22, wherein the surface area of the platform is smaller than the surface area of the MCU, thereby allowing free access to the front panel of the MCU.

32. The system of claim 22, configured to facilitate placement and securing of the MCU to the platform in a reversible manner.

33. (canceled)

34. (canceled)

35. The system of claim 22, configured to facilitate uninterrupted physical connection of the endoscope to the MCU and configured to facilitate interpreted operation of the endoscope during a medical procedure, while the endoscope is connected to the MCU.

36. (canceled)

37. (canceled)

38. (canceled)
